(19) United States
(12) Patent Application Publication
(10) Pub. No.: US 2007/0032758 A1
(43) Pub. Date: Feb. 8, 2007

(54) Lubricated tampon applicator

(76) Inventors: David J. Chase, Somerville, NJ (US); Tara Glasgow, New Hope, PA (US); Raymond J. Hull JR., Hampton, NJ (US); Stephen J. Mohary, Pennington, NJ (US); Linda M. Pierson, Stockton, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/393,573

(22) Filed: Mar. 30, 2006

Related U.S. Application Data
(60) Provisional application No. 60/667,341, filed on Mar. 31, 2005.

(51) Int. Cl. A61F 13/20

(52) U.S. Cl. 604/12; 604/15

(57) ABSTRACT

An applicator includes a barrel having an outer surface, an insertion end, and an opposed gripper end; a fluid lubricant disposed in fluid communication with a portion of the outer surface of the barrel; and optionally a plunger arranged and configured to be moveable within the barrel to expel an insertable device from the barrel and into a body cavity. Methods of delivering an insertable device into a body cavity may include the steps of moving a plunger into an insertion member to dispense fluid lubricant to the outer surface of the insertion member; the step of inserting an insertion member portion having encapsulated lubricant on an outer surface thereof into a body cavity thereby rupturing the capsule; and/or the steps of moving a plunger into an insertion member, rupturing a reservoir containing fluid lubricant, moving the fluid lubricant toward the outer surface of the insertion member, and inserting the insertion member into the body cavity.

### CROSS-REFERENCE TO RELATED APPLICATIONS

This invention is related to the following copending application: U.S. Ser. No. 11/______, filed on even date herewith, entitled “Novel Tampon Applicator” (Att'y Docket, J&J-5168).

### FIELD OF THE INVENTION

The present invention relates to applicators for delivering materials into mammalian body cavities. The applicator is particularly useful for delivering intravaginal devices, such as catamenial devices, into a vaginal canal.

### BACKGROUND OF THE INVENTION

Applicators for delivering materials into a body cavity typically comprise a tubular insertion member having an insertion end and a gripper end opposite thereof, and an elongate expulsion member slideably fitted within the tubular insertion member for expelling the contained materials.

### SUMMARY OF THE INVENTION

The present invention relates to an applicator for delivering an insertable device into a body cavity. The applicator includes a barrel, a fluid lubricant, and optionally a plunger arranged and configured to be moveable within the barrel to expel an insertable device from the barrel and into a body cavity. The barrel has an outer surface, an insertion end, and an opposed gripper end. The fluid lubricant is disposed in fluid communication with at least a portion of the outer surface of the barrel. The fluid lubricant may be disposed within the barrel, and/or contained within a reservoir. Preferably, the barrel has at least one fluid conduit disposed between the outer surface of the barrel and an inner surface of the barrel disposed toward the insertable device useful to deliver the fluid lubricant to the outer surface of the barrel. Additionally, the outer surface of the barrel defines apertures in fluid communication with the at least one fluid conduit.

Additionally, the present invention relates to methods of delivering an insertable device into a body cavity. For example, a method comprising the steps of moving a plunger into an insertion member dispenses fluid lubricant to the outer surface of the insertion member. Alternatively, a lubricant may be encapsulated on the outer surface of the insertion member; upon insertion into the body cavity, the capsules rupture to dispense lubricant. Yet another embodiment, the steps of moving a plunger into an insertion member ruptures a reservoir to move fluid lubricant toward the outer surface of the insertion member insertion.

### BRIEF DESCRIPTION OF THE DRAWING

FIG. 1A is a perspective view of an applicator according to the present invention comprising a tubular insertion member and a lubricant carrier disposed thereon.

FIG. 1B is a cross-section along line B-B of FIG. 1A.

FIG. 2 is a longitudinal cross-section of an applicator, such as depicted in FIG. 1A, having a reservoir connected to the insertion member and an exploded view of a portion of the insertion member.

FIG. 3 is a longitudinal cross-section of an applicator, such as depicted in FIG. 1A, having a reservoir disposed between and defined by the insertion member and plunger and an exploded view of a portion of the insertion member.

FIGS. 4A-C, are longitudinal cross-sections of an applicator, such as depicted in FIG. 1A, having a reservoir disposed between the plunger and an insertable device, and FIG. 4B includes an exploded view of a portion of the insertion member.

FIGS. 5A-B are transverse cross-sections of an applicator wherein the reservoir is formed of a plurality of discrete sections disposed between the insertion member and plunger.

### DETAILED DESCRIPTION OF THE INVENTION

The term “diameter” as used in herein the specification and the claims means a chord passing through the center of a figure or body; the length of a straight line through the center of an object

As used herein the specification and the claims, the term “intravaginal device” and related terms includes support devices, obstructing devices useful to block the flow of and/or collect bodily liquids, and the like. The term includes, without limitation, incontinence devices and vaginal supports, such as pessaries; and obstructing devices, such as menstrual collection cups and inflatable or expandable vaginal blocking devices (devices which do not, themselves, absorb the bodily liquids).

While the present invention generally relates to applicator devices having a tubular insertion member, the following detailed description will refer, specifically, to a tampon applicator for ease of understanding. One of ordinary skill in the art will recognize other uses for this invention including, without limitation, applicators for other catamenial devices, such as collection cups, inflatable plugs or cups, and the like; incontinence devices; suppositories; nasal packing; and the like.

The present invention relates to applicators for delivering materials into body cavities, comprising an elongate insertion member for at least partial insertion into a body cavity. The elongate insertion member has an insertion end and gripper end opposite thereof.

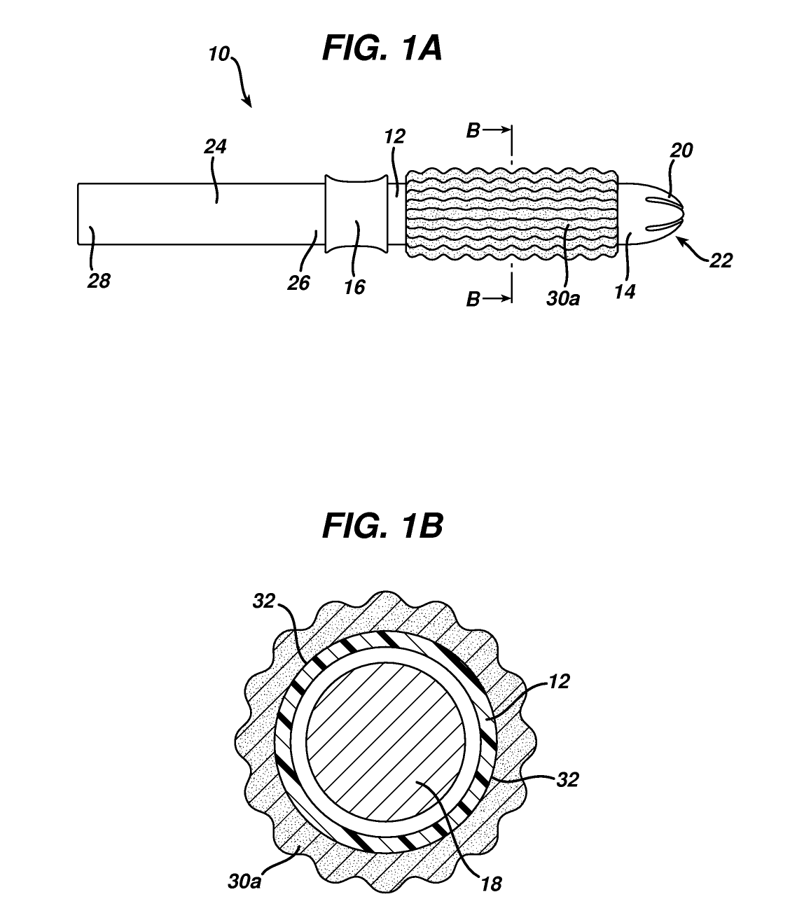

Referring now to the drawings, wherein like-reference numerals designate like elements, FIG. 1 depicts an applicator 10, comprising a tubular insertion member 12, having an insertion end 14 and a gripper end 16 that is suitable to contain an insertable device 18 for the body cavity. The insertion end 14 may have a plurality of inwardly curved petals 20 that form a substantially closed feature 22, and the gripper end 16 may have a structure 17 to enhance the user's grip on the applicator. The applicator 10 also includes an expulsion member 24 having a first end 26 that is insertable into the tubular insertion member 12 and is capable of bearing against the device 18. The expulsion member 24 terminates in a second end 28, opposite the first end 26, that may be manipulated to move the first end 26 within the tubular insertion member 12. The first end 26 of the expulsion member 24 is arranged and configured for slideable introduction into the tubular insertion member 12 through its gripper end 16.

The applicators 10 or other tubular devices of the present invention can have tube geometries or cross-sections that are useful to contain the object to be inserted. Often, the shape of the insertable device 18 contained suggests the shape of the tubular insertion member 12, but departures from this general rule may be made. Therefore, the tubular insertion member 12 may take on numerous cross-sectional shapes including, without limitation, circular, oval, polygonal (e.g., trapezoidal, rectangular, triangular), and the like. For example, cylindrical tampons may be contained within rectangular insertion members and trapezoidal tampons (such as those disclosed in Van Iten et al., U.S. Pat. No. 5,350,371) and cup-shaped tampons (such as those disclosed in Bailey, U.S. Pat. No. 2,330,257) can be contained in a generally cylindrical insertion member. In addition, the insertion member 12 can substantially elongated, curved, or flexible, or it can take on other shapes that are apparent to one of ordinary skill in the art. The specific geometry, itself, is not critical to the practice of the present invention. In addition, the edge of the tubular device (both finished and unfinished) may be a standard, planar edge coincident with a plane perpendicular to the longitudinal axis of the tubular device.

The applicator devices of the present invention can be made of materials known to those of ordinary skill in the art. Generally, the applicators may be plastic or paper. Plastic materials include, without limitation, polyolefins such as polyethylene and polypropylene, polyesters such as polyethylene terephthalate, polycaprolactone, polyvinyl alcohol, ethylene-vinyl acetate copolymers, cellophane, PHBV such as those disclosed in Dabi et al., U.S. Pat. No. 5,910,520 (herein incorporated by reference), starch-based polymers including those disclosed in Dabi et al., U.S. Pat. No. 5,910,520, and the like. The expulsion member can be formed as a solid or a tubular element.

Paper materials include, without limitation, paperboard, cardboard, cup stock, paper, and the like. The paper may be a single layer of material, or it can be a plurality of laminated layers to provide multiple benefits relating to the various layers. Laminated paper material may include a surface layer or coating of plastic, wax, silicone, lubricants, and the like, which may be useful to increase the comfort to the user during insertion and withdrawal. The plastic coating may include, without limitation, those plastic materials listed above. Laminated paper material may also include additional layers such as adhesive layers, tie layers, and the like.

An example of such a surface layer is disclosed in Blanchard, U.S. Pat. No. 6,171,426. A representative, non-limiting list of useful materials to be used as the surface layer includes, waxes, cellophane, polyolefins, polyesters, epoxies, and the like. The surface layers may also include thermal stabilizers, pigments, fragrances, surfactants, antimicrobial agents, medicaments, and the like.

Typical dimensions for each of the tubular insertion and expulsion members include a length of from about 50 to about 100 millimeters, a diameter of from about 8 to about 16 millimeters, and a thickness of from about 0.4 to about 0.6 millimeters. Preferably, the diameter of the expulsion member is less than the diameter of the tubular insertion member to allow for a telescopic arrangement of the two.

The tubular insertion member of the applicator provided by the present invention is preferably substantially closed prior to expulsion of the materials contained therein. Alternatively, the insertion end of the applicator can be more or less open, that is the diameter along the length of the tubular insertion member is substantially equivalent to the diameter of the insertion end. Procter & Gamble, of Cincinnati, Ohio, currently offers for sale an open-ended tampon applicator under the trade name TAMPAX® flushable applicator tampons.

In order to facilitate insertion and removal of the applicator from the body, a fluid lubricant can be delivered to the applicator surface that may have a lubricant carrier (generically “30”) operatively linked thereto. The fluid lubricant may also provide cleansing and/or freshness to the user. As used herein the Specification and Claims, the term “operatively linked” relates to elements that are connected directly or indirectly to each other. For example, a lubricant carrier affixed to the applicator would be operatively linked. Alternatively, a lubricant carrier that is not physically attached to the applicator, but which is attached to an insertable device contained within the applicator would also be operatively linked to the applicator.

Referring to FIGS. 1A and 1B, the lubricant carrier 30a can be a textured pile fabric, such as terry cloth, or other textured fibrous structure. In this embodiment, the lubricant carrier 30a is affixed to the tubular insertion member 12 about its outer surface 32. As shown in FIG. 1A, the lubricant carrier 30a extends from the insertion end to the gripper end (as shown, both the extreme insertion and gripper ends are left uncovered by the lubricant carrier 30a).

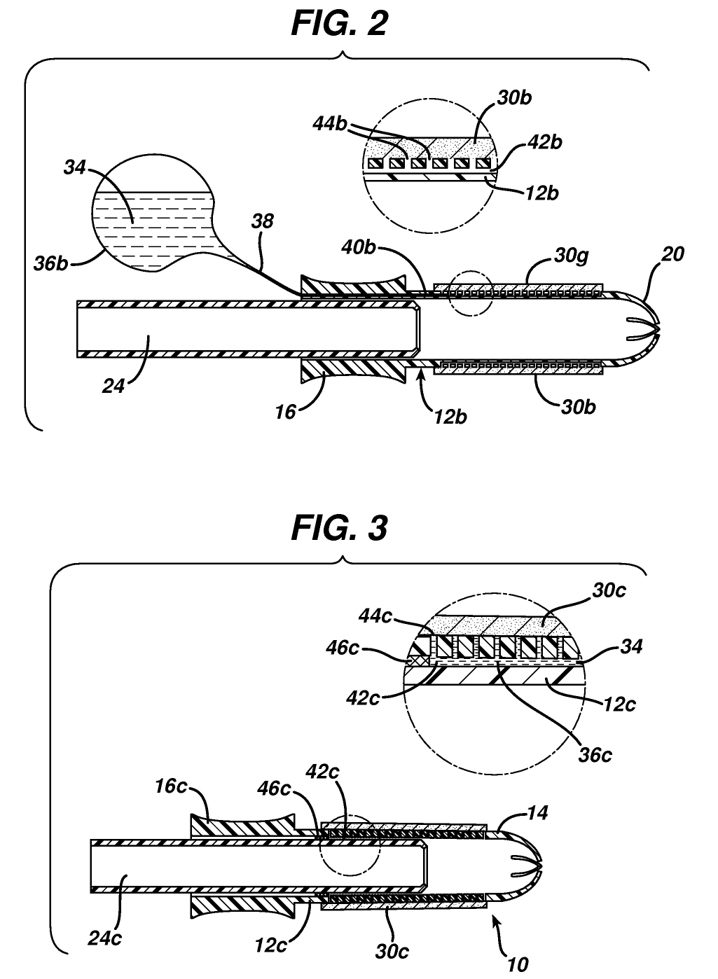

According to the present invention, a fluid lubricant 34 is not disposed on or within a lubricant carrier 30b. Instead, the fluid lubricant 46 is preferably contained within a reservoir 48b. Referring to FIG. 2, a reservoir 48b, e.g., a squeeze bulb, is connected to the insertion member 12 through a tube 38b. The insertion member 12b has formed in the walls thereof a conduit 40b and manifold 42b that distributes the fluid lubricant 34 through a plurality of apertures 44b formed in the outer surface 32b of the insertion member 12b. The fluid lubricant 34 then may permeate the fluid carrier 30b.

The lubricant carrier 30 may also “swab” the surfaces of the body cavity into which it is inserted, during both insertion and withdrawal motions. This wiping or cleansing action is helpful to provide a clean and fresh feel to the user. Therefore, lubricant may also incorporate additives. A representative, non-limiting list of such additives includes cleansers, emollients, anti-inflamatories, probiotics, antibiotics, and the like. Specific compositions may include health-promoting compositions such as glyceryl monolaurate as disclosed in Brown-Skrobot, U.S. Pat. No. 5,641,503, the disclosure of which is herein incorporated by reference.

Referring to FIG. 3, a reservoir 36c is defined by the insertion member 12c and plunger 24c. The insertion member 12c is tapered from a relatively large diameter proximate the gripper end 16c toward a smaller diameter proximate the insertion end 14c. As the plunger 24c is moved into the insertion member 12c, a resilient ring 46c moves the fluid lubricant 34 along the inner surface 48 of the insertion member 12c. As the annular space between the inner surface 48 and the plunger cylinder 50c diminishes, the fluid lubricant 34 is forced through the apertures 44c and to the outer surface 32c of the insertion member 12c. The fluid lubricant 34 thus coats the outer surface 32c or saturates a lubricant carrier 30c, depending upon the structure of the applicator 10.

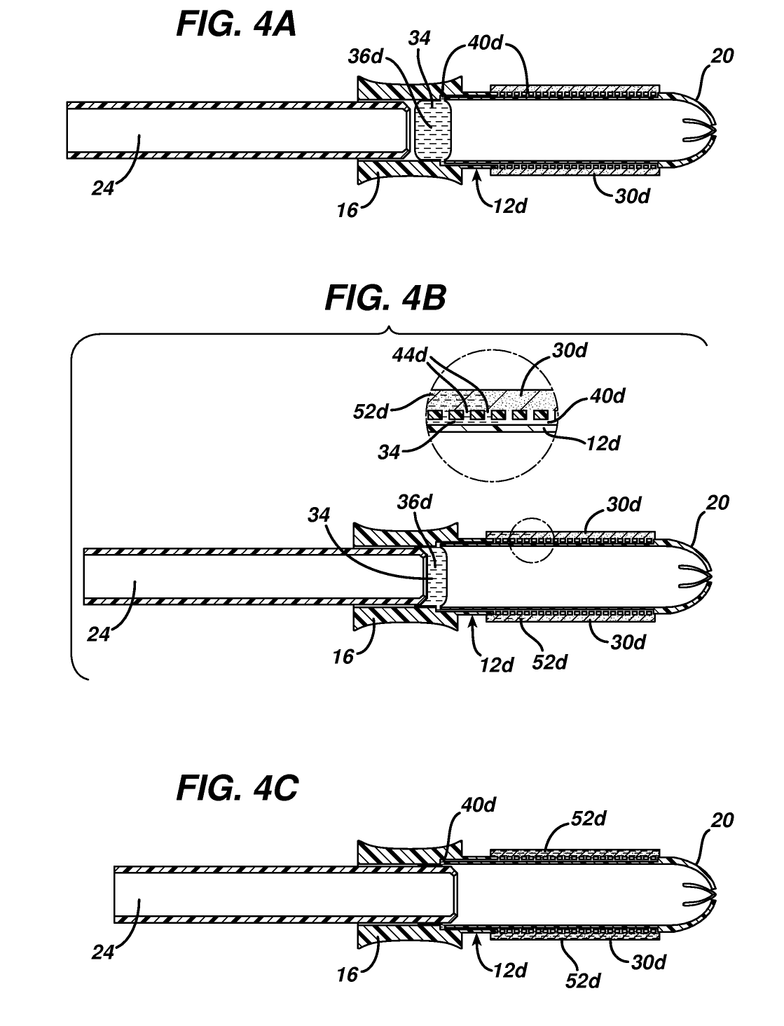

Referring to FIGS. 4A-C, a reservoir 36d is disposed between the plunger 24 and insertable device 18. The reservoir 36d is in fluid communication with the outer surface 32d of the insertion member 12d, e.g., through a conduit 40d. As the plunger 24 advances within the insertion member 12d, the plunger forces the fluid lubricant 34 from the reservoir 36d, into the conduit 40d, through apertures 44d, and into lubricant carrier 30d to form at least one saturated region 52d. As the plunger 24 continues into the insertion member 12d, substantially all of the lubricant carrier 30d may become saturated, and the plunger can destroy or otherwise bypass or pass through the reservoir 36d and eject the insertable device 18.

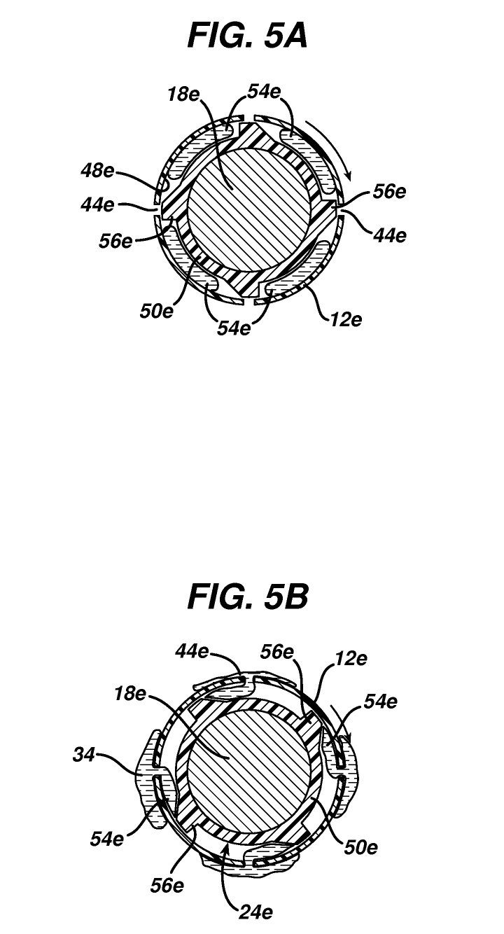

Referring to FIGS. 5A-B, a reservoir 36e may be formed of a plurality of discrete sections 54e, each of which may be associated with one or more apertures 44e. FIG. 5A shows the applicator system prior to use. Apertures 44e are closed by plunger projections 56e that extend radially outwardly from the plunger cylinder 50e. The discrete sections 54e may be arranged between the plunger cylinder 50e and the inner surface of the insertion member 48e. Thus, when the plunger 24e is rotated, e.g., clockwise (in the direction of the arrow), an inclined face of the projections 56e can direct the fluid lubricant 34 through the now-opened apertures 44e to coat the outer surface 32e of the insertion member 12e. The outer surface 32e of the insertion member 12e, may optionally have a separate lubricant carrier (not shown) disposed thereon.

The applicator of the present invention can be constructed of paperboard or plastic. The paperboard applicators can be constructed from a single layer of paper material, or from a plurality of laminated layers to provide multiple benefits relating to the various layers. The applicators can be made from sheets of material using several processing including, without limitation: spiral winding as disclosed in Campion et al., U.S. Pat. No. 5,346,468, convolute winding as disclosed in Whitehead, U.S. Pat. No. 4,508,531, and forming a sheet around a mandrel and then sealing an overlapped seam as disclosed in Hinzmann, U.S. Pat. No. 4,755,164.

If the applicator includes a surface layer, the layer may be applied using any useful technique. Many techniques are known for applying the surface layers. A representative non-limiting list of such techniques includes spraying, extruding, slot-coating, brushing, transfer coating, and the like. Additional processing steps may be required to cure the surface treatments to a useable form other than simple air curing, such as applying irradiation or other forms of energy.

The insertion member of the applicator provided by the present invention is preferably substantially closed prior to expulsion of the materials contained therein. One technique for substantially closing the insertion end of the applicator is by employing a plurality of inwardly curved petals. The petals will flex and/or hinge to an open position upon expelling materials contained by the applicator. The number of petals generally ranges from about 4 to about 6. An alternative technique for substantially closing the insertion end of an applicator is by pleating the insertion end. This technique is disclosed in Neilsen et al., U.S. Pat. No. 5,782,793. When an applicator is constructed with more than one layer of material, a single layer may extend into the insertion end in an effort to reduce the force required to expel the contained materials. An example of this is disclosed in Fox et al., U.S. Pat. No. 5,827,214. These collective closures may be of spherical shape, or alternatively tapered shape.

Plastic applicators may be manufactured using any useful technique. Many techniques are known for manufacturing plastic applicators. A representative, non-limiting list of such techniques includes injection-molding, blow-molding, extrusion, formation from one or more sheets (as described above for paper), and the like. Generally, at least the tubular insertion members can be formed through an injection molding process. This process is often used, because it allows the manufacture to balance some key characteristics of the tubular insertion member. Molding inserts and cores can be machined to form a slightly tapered product. For example, the wall thickness around the gripping end is relatively thick to maintain structural stability during the insertion and expulsion steps of use, while the thickness in the insertion end can be minimized to provide flexibility and low expulsion force. Injection molding also enables the manufacture to make uniquely shaped tubular insertion members. As mentioned above, the less sophisticated and/or less expensive techniques, such as extrusion and blow molding can also be employed.

The intrawall conduits described above relating to FIGS. 8-11 can be more easily formed in plastic applicators. The appropriate number and form of channels can be formed through procedures known to those of ordinary skill in the art. In addition to forming the channels during molding, an open channel in the outer surface of an insertion member can be covered with a film or other outer member to enclose the channel.

The applicator of the present invention can be used for the delivery of an object into a mammalian body cavity. Such objects may include suppositories, absorbent devices, and the like, and they may be delivered into body cavities including the mouth, nose, vagina, and rectum. These materials may be in the form of solids, creams, foams, gels, and the like.

Preferably, the applicator is used to deliver intravaginal devices, including catamenial devices, such as tampons, intravaginal collection devices, and interlabial pads; birth control devices such as diaphragms or intrauterine devices (IUDs); compositions in the form of suppositories, such as medicaments, moisturizers, vitamins and minerals, spermicides, and odor controlling agents; medical devices and incontinence devices and vaginal supports such as pessaries; and obstructing devices. Obstructing devices include menstrual collection cups and inflatable or expandable blocking devices.

In use, a woman may place the insertion end into the body cavity orifice, delivering the insertable device into the body cavity by pushing on expulsion member until the insertable device is expelled from the tubular insertion member and withdrawing applicator from the body, leaving insertable device within the body cavity.

Alternately, a user could pull tubular insertion member onto expulsion member while maintaining expulsion member steady relative the user's body. This substantially eliminates friction between the insertable device and the user's body.

What is claimed is:

1. An applicator for delivering an insertable device into a body cavity comprising
a. a barrel having an outer surface, an insertion end, and an opposed gripper end; and
b. a fluid lubricant disposed in fluid communication with at least a portion of the outer surface of the barrel

2. The applicator of claim 1, further comprising a plunger arranged and configured to be moveable within the barrel to expel an insertable device from the barrel and into a body cavity.

3. The applicator of claim 1, wherein the fluid lubricant is disposed within the barrel.

4. The applicator of claim 1, wherein the fluid lubricant is contained within a reservoir.

5. The applicator of claim 1, wherein the barrel has at least one fluid conduit disposed between the outer surface of the barrel and an inner surface of the barrel disposed toward the insertable device useful to deliver the fluid lubricant to the outer surface of the barrel.

6. The applicator of claim 5, wherein the outer surface of the barrel defines apertures in fluid communication with the at least one fluid conduit.

7. A method of delivering an insertable device into a body cavity comprising the steps of
a. moving a plunger into an insertion member, the insertion member
i. containing the insertable device and
ii. having an outer surface to dispense fluid lubricant to the outer surface of the insertion member;
b. inserting the insertion member into a body cavity; and
c. moving the plunger to deliver the insertable device into the body cavity.

8. A method of delivering an insertable device into a body cavity comprising the steps of
a. inserting an insertion member portion of a device applicator into a body cavity, the insertion member portion having lubricant contained in at least one capsule on an outer surface thereof and containing the insertable device;
b. rupturing the at least one capsule to dispense lubricant; and
c. delivering the insertable device into the body cavity.

9. A method of delivering an insertable device into a body cavity comprising the steps of
a. moving a plunger into an insertion member having an outer surface and containing the insertable device;
b. rupturing at least a portion of a reservoir containing fluid lubricant;
c. moving the fluid lubricant toward the outer surface of the insertion member;
d. inserting the insertion member into a body cavity; and
e. moving the plunger to deliver the insertable device into the body cavity.
